(19) United States
(12) Patent Application Publication
(10) Pub. No.: US 2025/0020590 A1
(43) Pub. Date: Jan. 16, 2025

(54) SYSTEM FOR DISCRIMINATING ECO-FRIENDLY MATERIAL FOR A VEHICLE AND A METHOD OF DISCRIMINATING ECO-FRIENDLY MATERIAL FOR A VEHICLE USING SAME

(71) Applicants: HYUNDAI MOTOR COMPANY, Seoul (KR); KIA CORPORATION, Seoul (KR)

(72) Inventor: Chae-Hwan Hong, Seoul (KR)

(73) Assignees: HYUNDAI MOTOR COMPANY, Seoul (KR); KIA CORPORATION, Seoul (KR)

(21) Appl. No.: 18/376,329

(22) Filed: Oct. 3, 2023

(30) Foreign Application Priority Data
Jul. 13, 2023 (KR) 10-2023-0091045

(51) Int. Cl. G01N 21/64; G01N 33/44

(52) U.S. Cl. CPC G01N 21/64; G01N 33/442

(57) ABSTRACT

A system for discriminating an eco-friendly material for a vehicle, and a method of discriminating an eco-friendly material for a vehicle using the same is disclosed. More specifically, through the system for discriminating an eco-friendly material for a vehicle by using fluorescence spectroscopic analysis on an eco-friendly material for a vehicle, which can be discriminated and is made of a composite in which a fluorescent material with a unique emission characteristic and a plastic material are mixed, the discrimination method quickly and reliably discriminates an eco-friendly plastic material for a vehicle from fluorescence and spectral characteristics of the fluorescent material.

### CROSS-REFERENCE(S) TO RELATED APPLICATIONS

This application claims priority to Korean Patent Application No. 10-2023-0091045, filed on Jul. 13, 2023, which is incorporated herein by reference in its entirety.

### BACKGROUND OF PRESENT DISCLOSURE

### Technical Field

Embodiments of the present disclosure relate to a system for discriminating an eco-friendly material for a vehicle, which can simply discriminate information on recycling of an eco-friendly plastic material for a vehicle or information to which an eco-friendly manufacturing method is applied from a material and parts distribution process and a supply chain, and a method of discriminating an eco-friendly material for a vehicle using the same.

### Description of Related Art

Currently, the amount of plastic waste generated in Korea is showing an annual average increase of about 50%, but the generated plastic waste is not effectively recycled and more than 50% thereof is incinerated or landfilled. However, the incineration and landfill of the plastic waste pollutes water and air to causes serious environmental problems, and the interest in recycling of the plastic waste is increasing day by day.

Some of the plastic waste is recycled, but most of the plastic waste ends up in landfills, and only some is used in waste combustion plants and converted into thermal energy. This approach is a clear waste of valuable raw materials that could be effectively used again and contributes to increased emissions of greenhouse gases into the atmosphere. In particular, the recycling of plastic materials generated in the automobile industry is an important issue in the automobile sector that aims for carbon neutrality.

Therefore, in the entire process from automobile production to scrapping, the technology in which used plastic materials are reused can be expected to reduce carbon emissions. However, there is a problem in that one of the industrially important issues is whether determining whether the plastic materials are eco-friendly materials should depend only on the manufacturer's information. Whether other parties know detailed information or not or the reliability of the information provided from the manufacturer is low in some cases. In other words, an effective discrimination method is not established at the stage of manufacturing plastic parts such as extrusion and injection by manufacturing materials and utilizing the materials.

### SUMMARY

An embodiment of the present disclosure is directed to providing a system for discriminating an eco-friendly material for a vehicle, which is capable of discriminating a plastic material by using a photoluminescence method in which a plastic material for a vehicle and a specific fluorescent material are mixed, and light emission (luminescence), which does not accompany heat by obtaining light energy from the outside, is measured. Another embodiment of the present disclosure is directed to a method of discriminating an eco-friendly material for a vehicle using the same.

Other objects and advantages of the present disclosure can be understood by the following description and become apparent with reference to the embodiments of the present disclosure. Also, it is understood to those having ordinary skill in the art to which the present disclosure pertains that the objects and advantages of the present disclosure can be realized by the means as claimed and combinations thereof.

In accordance with an embodiment of the present disclosure, there is provided a system for discriminating an eco-friendly material for a vehicle. The system includes: a fluorescent material supply part configured to store and supply a fluorescent material with a unique emission characteristic when irradiated with light; a plastic supply part configured to store and supply a plastic material; a composite compounding part configured to manufacture a composite by mixing and melting the fluorescent material supplied from the fluorescent material supply part and the plastic material supplied from the plastic supply part and extrude the manufactured composite; a molding part configured to manufacture a plastic molded product by performing an injection molding process on the composite manufactured in the composite compounding part; and a discrimination part configured to measure a fluorescence intensity from the composite or the plastic molded product.

In the system for discriminating an eco-friendly material for a vehicle, the fluorescent material may use one or more of acridine, anthracene, coumarin, cyanine, stilbene-based compounds, derivatives thereof, or combinations thereof. In addition, the fluorescent material may be included in an amount ranging from 0.1 wt % (percentage by weight) to 10 wt % based on the total weight of the composite.

In the system for discriminating an eco-friendly material for a vehicle, the composite or the plastic material may include one or more of polypropylene (PP), nylon, polycarbonate (PC), acrylonitrile butadiene styrene (ABS), polyethylene terephthalate (PET), or combinations thereof.

In the system for discriminating an eco-friendly material for a vehicle, the composite compounding part may manufacture the composite by mixing and melting the fluorescent material and the plastic material at a temperature ranging from 200° C. to 270° C.

In the system for discriminating an eco-friendly material for a vehicle, the discrimination part may measure a value of a fluorescence intensity emitted from the composite or the plastic molded product by irradiating the composite or the plastic molded product with light with a wavelength ranging from 190 nm to 900 nm, analyze the measured value of the fluorescence intensity, and discriminate whether an eco-friendly material is used.

In accordance with another embodiment of the present disclosure, there is provided a method of discriminating an eco-friendly material for a vehicle. The method includes (a) irradiating a composite or plastic molded product to be measured with light and measuring a value of a fluorescence intensity emitted therefrom, and (b) determining whether the composite or the plastic molded product is a recycled plastic material from the measured value of the fluorescence intensity.

In the method of discriminating an eco-friendly material for a vehicle, in operation (a), a value of an emitted fluorescence intensity may be measured through a fluorescence spectrophotometer by irradiating the composite or the plastic molded product with light with a wavelength ranging from 190 nm to 900 nm.

In the method of discriminating an eco-friendly material for a vehicle, in operation (b), whether the composite or the plastic molded product is the recycled plastic material may be determined by comparing the value of the fluorescence intensity measured from the composite or the plastic molded product with a pre-stored value of a fluorescence intensity of a standard material.

The standard material may be a plastic material not containing a fluorescent material with a unique emission characteristic when irradiated with light.

### BRIEF DESCRIPTION OF THE DRAWINGS

FIG. 1 is a schematic block diagram illustrating a system for discriminating an eco-friendly material for a vehicle according to an embodiment of the present disclosure.

FIG. 2 is a mimetic diagram illustrating a fluorescence phenomenon in a fluorescence spectrophotometer according to an embodiment of the present disclosure.

FIG. 3 is a graph showing fluorescence analysis results of composites of Examples 1 to 4 according to an embodiment of the present disclosure.

### DESCRIPTION OF SPECIFIC EMBODIMENTS

The above and other objectives, features, and advantages of the present disclosure should become more apparent from the following description of embodiments with reference to the accompanying drawings. However, the present disclosure is not limited to the embodiments disclosed herein and may be implemented in other forms. Rather, the embodiments disclosed herein are provided to make this disclosure thorough and complete and fully convey the spirit of the present disclosure to those having ordinary skill in the art.

In the present specification, the terms “comprising,” “having,” or the like are used to specify that a feature, a number, a step, an operation, a component, an element, or a combination thereof described herein exists, and they do not preclude the presence or addition of one or more other features, numbers, steps, operations, components, elements, or combinations thereof.

Unless otherwise specified, all numbers, values, and/or expressions indicating ingredients, reaction conditions, and quantities of plastic and a fluorescent material used herein are approximations to which various uncertainties of measurement are reflected, wherein the various uncertainties occur in obtaining these values among essentially different other things so that it should be understood that all numbers, values, and/or expressions are modified by a term “about.”

In addition, when a numerical range is disclosed herein, such a numerical range is continuous and, unless otherwise indicated, the numerical range includes all values from a minimum value to a maximum value. Further, when the numerical range refers to integers, unless otherwise indicated, all integers from a minimum value to a maximum value are included.

In the present specification, when a range is described for a variable, it is understood that the variable includes all values within the range including endpoints described in the range. For example, it is understood that a range from “5 to 10” includes values of 5, 6, 7, 8, 9, and 10 as well as any subranges such as ranges from 6 to 10, from 7 to 10, from 6 to 9, from 7 to 9, and the like, and also includes any values between integers, which are reasonable in the scope of ranges, such as 5.5, 6.5, 7.5, 5.5 to 8.5, and 6.5 to 9, and the like. In addition, for example, it is understood that a range from “10% to 30%” includes all integers including values 10%, 11%, 12%, 13%, and the like and up to 30% as well as subranges from 10% to 15%, from 12% to 18%, from 20% to 30%, and the like, and also includes any values between integers, which are reasonable in the scope of ranges, such as 10.5%, 15.5%, 25.5%, and the like.

When a component, device, element, or the like of the present disclosure is described as having a purpose or performing an operation, function, or the like, the component, device, or element should be considered herein as being “configured to” meet that purpose or to perform that operation or function.

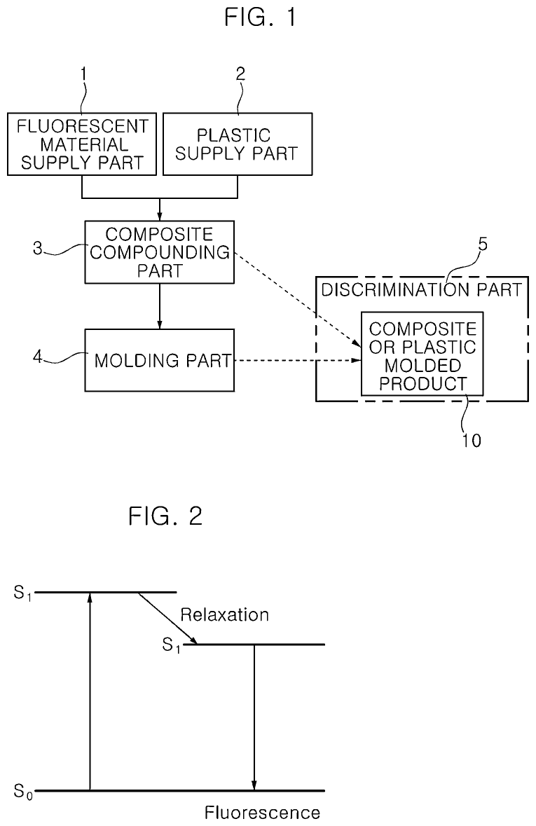

FIG. 1 is a schematic block diagram illustrating a system for discriminating an eco-friendly material for a vehicle according to an embodiment of the present disclosure.

As shown in FIG. 1, the system for discriminating an eco-friendly material for a vehicle of the present disclosure includes a fluorescent material supply part 1, a plastic supply part 2, a composite compounding part 3, a molding part 4, and a discrimination part 5.

The fluorescent material supply part 1 stores and supplies a fluorescent material to be added to a plastic material. Specifically, the fluorescent material supply part 1 is a device that is filled with nitrogen gas, is provided with a material transfer screw installed therein, and stores and supplies a fluorescent material of a cone style shape.

In one example, at least one material comprising acridine, anthracene, coumarin, cyanine, stilbene-based compounds, and/or derivatives or combinations thereof is used as the fluorescent material.

The acridine derivative is, for example, acridine homodimer (CAS number is 57576-49-5, and a molecular formula is C₃₈H₄₂C₁₂N₆O₂), acridine orange (CAS number is 65-61-2, and a molecular formula is C₁₇H₂₀ClN₃), or acridine orange 10-dodecyl bromide (CAS number is 41387-42-2, and a molecular formula is C₂₉H₄₄BrN₃).

The anthracene derivative is, for example, 9-anthroylnitrile (CAS Number is 85985-44-0, and a molecular formula is C₁₆H₉NO).

The coumarin derivative is, for example, 6,7-dimethoxycoumarin-4-acetic acid (CAS number is 88404-26-6, and a molecular formula is C₁₃H₁₂O₆), 7-dimethylaminocoumarin-4-acetic acid (CAS number is 80883-54-1, and a molecular formula is C₁₃H₁₃NO₄), or 7-dimethylamino-4-methylcoumarin-3-isothiocyanate (CAS number is 74802-04-3, and a molecular formula is C₁₃H₁₂N₂O₂S).

The cyanine derivative is, for example, 3,3-diethyloxacarbocyanine iodide (CAS number is 905-96-4, and a molecular formula is C₂₁H₂₁IN₂O₂), 3,3-dihepthyloxacarbocyanine iodide (CAS number is 53213-83-5, and a molecular formula is C₃₁H₄₁IN₂O₂), or 3,3-dihexyloxacarbocyanine iodide (CAS number is 53213-82-4, and a molecular formula is C₂₉H₃₇IN₂O₂).

The stilbene derivative is, for example, trans-stilbene (CAS number is 103-30-0, and a molecular formula is C₁₄H₁₂), 4-dimethylamino-4-nitrostilbene (CAS number is 2844-15-7, and a molecular formula is (CH₃)₂NC₆H₄CH═CHC₆H₄NO₂), or 4,4′-bis(2-benzoxazolyl)stilbene (CAS number is 1533-45-5, and a molecular formula is C₂₈H₁₈N₂O₂).

The plastic supply part 2 stores and supplies a plastic material to be used.

The plastic material may include one or more materials comprising polypropylene (PP), nylon, polycarbonate (PC), acrylonitrile butadiene styrene (ABS), polyethylene terephthalate (PET), or a combination thereof. The plastic material is a plastic material generally used for vehicles, and materials used for interior and exterior parts and engine chassis parts may be used as the plastic material.

The plastic material is waste plastic to be recycled, and any waste plastic to be disposed of may be freely used.

The composite compounding part 3 manufactures a discriminable eco-friendly material for a vehicle and may be a melting extrusion device for mixing and melting the fluorescent material supplied from the fluorescent material supply part 1 and the plastic material supplied from the plastic supply part 2 to manufacture a composite and extrude the manufactured composite.

In an embodiment of the present disclosure, the composite is referred to as a composite material and means a material manufactured by mixing the plastic material and the fluorescent material together.

The melting extrusion device of the composite compounding part 3 according to one embodiment includes a pair of extrusion gears for melting and extruding an introduced plastic material by rotating while engaging with each other.

The melting extrusion device of the composite compounding part 3 may include a supply part for supplying a predetermined amount of the fluorescent material (from the fluorescent material supply part 1) to the plastic material (from the plastic supply part 2) before the melting and extrusion of the plastic material is performed.

In one example, a content of the fluorescent material to be added ranges from 0.1 wt % to 10 wt % based on the total weight of the eco-friendly material for a vehicle. Contents in the range of 0.3 wt % to 5 wt % may be more appropriate.

When the content of the fluorescent material is less than 0.1 wt %, there is a problem in that sensitivity of fluorescence analysis is degraded due to the small content and discrimination is poor. On the other hand, when the content of the fluorescent material exceeds 10 wt %, there is a problem in that separation on fluorescence spectrum deteriorates due to the excessive content. Thus, it is beneficial to satisfy the range suggested above.

In one example, the composite compounding part 3 may mix and melt the fluorescent material and the plastic material at a temperature ranging from 200° C. to 270° C.

When the melting temperature is less than 200° C., the plastic material may not be melted properly due to the low temperature, and thus the fluorescent material may not be uniformly dispersed. On the other hand, when the melting temperature exceeds 270° C., which is a too high temperature, composite production efficiency and energy efficiency are entirely degraded. Thus, it is beneficial to perform the melting in the temperature range suggested above.

Before extruding the composite in a molten state to the outside, the composite compounding part 3 may perform a process of stabilizing a state of the composite by mixing and melting the fluorescent material and the plastic material and then staying (i.e., maintaining or retaining a current state) for a certain period of time in a state where a temperature condition is satisfied. In one embodiment of the present disclosure, it is beneficial to stabilize the molten composite by staying for 5 to 60 seconds.

The molding part 4 manufactures a plastic molded product through a molding process using the composite manufactured in the composite compounding part 3.

The discrimination part 5 analyzes the composite manufactured in the composite compounding part 3 or the plastic molded product manufactured in the molding part 4 (collectively shown as “composite or plastic molded product 10” in FIG. 1) using a fluorescence spectrophotometer to measure a value of a fluorescence intensity and confirm whether the composite or the plastic molded product 10 is a recycled plastic material through the presence or absence of a fluorescent material.

The discrimination part 5 irradiates the composite or the plastic molded product with light with a wavelength ranging from 190 nm to 900 nm from ultraviolet to visible rays, measures emitted fluorescence, and measures which wavelength emits how much strong fluorescence.

Since analysis reliability is degraded when the measurement wavelength range is out of the range of 190 nm to 900 nm presented above, it is beneficial to perform the measurement in the above range.

In the fluorescence spectrophotometer of the discrimination part 5, fluorescence is a phenomenon in which a molecule emits light with a wavelength that is longer than light with a short wavelength by absorbing the light with the short wavelength having high energy. As shown in FIG. 2, when a molecule absorbs light, the molecule rises to an excited state Si having higher energy. When the molecule absorbing the light rises to the excited state Si, the molecule becomes unstable and releases energy in the form of heat or emitted light with a different wavelength while falling back to its ground state S₀ so that fluorescence occurs.

Since the energy and wavelength of light absorbed and emitted are different depending on a type of material, various fluorescence spectra exhibit. Generally, a fluorescence spectrophotometer has something in common with a UV-Vis spectrophotometer in that a material absorbs light in a specific wavelength range by irradiating a material with light having a wavelength (ranging from 190 nm to 900 nm) in the range of ultraviolet to visible rays. However, there is a difference in that the UV-Vis spectrophotometer measures radiation not absorbed by the material to determine how much light is absorbed at which wavelength, whereas the fluorescence spectrophotometer measures fluorescence emitted from the material to determine which wavelength emits how much strong fluorescence.

A method of discriminating an eco-friendly material for a vehicle according to the present disclosure includes (a) irradiating a composite or plastic molded product 10 to be measured with light and measuring a value of a fluorescence intensity emitted therefrom, and (b) determining whether the composite or plastic molded product 10 is a recycled plastic material from the measured value of the fluorescence intensity.

In operation (a), a composite or plastic molded product 10 to be discriminated is accommodated or mounted in the discrimination part 5 of the eco-friendly material discrimination system and is irradiated with light with a wavelength ranging from 190 nm to 900 nm through a fluorescence spectrophotometer, thereby measuring a value of the emitted fluorescence intensity.

Next, in operation (b), the fluorescence (fluorescence intensity) value measured from the composite or plastic molded product 10 through operation (a) is analyzed to determine whether the composite or plastic molded product 10 is a recycled plastic material.

In operation (b), it is possible to determine whether the composite or plastic molded product 10 is the recycled plastic material by comparing the value of the fluorescence intensity measured from the composite or plastic molded product 10 with a value of a fluorescence intensity of a standard material, which is a plastic material not containing a pre-stored fluorescent material.

Hereinafter, the present disclosure is described in more detail through specific examples. The following examples are merely illustrative to aid understanding of the present disclosure, and the scope of the present disclosure is not limited by the following examples.

### EXAMPLE 1

In Example 1, the melt extrusion device of the composite compounding part 3, which was connected to the fluorescent material supply part 1 in which a particulate chemical 4,4-bis(2-benzoxazolyl) stilbene was stored as a fluorescent material and connected to the plastic supply part 2 in which a polypropylene plastic pelletized material was stored as a plastic material, operated at a specific rate (g/min) to transport each material. The fluorescent material was added to be included in an amount of 1 wt % based on the total weight of a composite, and the composite was manufactured by melting and mixing the fluorescent material and the plastic material under the condition in which a temperature of the melting extrusion device was 200° C. and a retention time of the molten composite was 5 seconds.

It was determined whether discrimination was possible by analyzing fluorescence of the manufactured composite using a fluorescence spectrometer (product model number FL 8500 by PerkinElmer). An operating condition of the fluorescence spectrometer was that a xenon arc lamp (150W) was applied, and a wavelength range of light incident on the material ranged from 200 nm to 900 nm.

### EXAMPLE 2

In Example 2, a composite was manufactured and fluorescence was analyzed in the same manner as in Example 1. However, a difference in manufacturing the composite is that the content of the fluorescent material in the composite was 3 wt %, the temperature of the melt extrusion device was 210° C., and the retention time of the melted composite was 10 seconds.

### EXAMPLE 3

In Example 3, a composite was manufactured and fluorescence was analyzed in the same manner as in Example 1. However, a difference in manufacturing the composite is that the content of the fluorescent material in the composite was 5 wt %, the temperature of the melt extrusion device was 220° C., and the retention time of the melted composite was 15 seconds.

### EXAMPLE 4

In Example 4, a composite was manufactured and fluorescence was analyzed in the same manner as in Example 1. However, a difference in manufacturing the composite is that the content of the fluorescent material in the composite was 7 wt %, the temperature of the melt extrusion device was 230° C., and the retention time of the melted composite was 20 seconds.

### Comparative Example 1

In Comparative Example 1, a composite was manufactured in the same manner as in Example 1 and fluorescence was analyzed, but a difference in manufacturing the composite is that magnetic nanocomposite particles, which were characterized in that a magnetic material is iron manganese, were used in an amount of 1 wt % instead of the fluorescent material in the composite.

### Comparative Example 2

In Comparative Example 2, a composite was manufactured in the same manner as in Example 2 and fluorescence was analyzed, but a difference in manufacturing the composite is that magnetic nanocomposite particles, which were characterized in that a magnetic material is iron manganese, were used in an amount of 3 wt % instead of the fluorescent material in the composite.

### Comparative Example 3

In Comparative Example 3, a composite was manufactured in the same manner as in Example 3 and fluorescence was analyzed, but a difference in manufacturing the composite is that 5 wt % magnetic nanocomposite particles, which was characterized in that a magnetic material is iron manganese, was used instead of the fluorescent material in the composite.

### Comparative Example 4

In Comparative Example 4, a composite was manufactured in the same manner as in Example 4 and fluorescence was analyzed, but a difference in manufacturing the composite is that 7 wt % magnetic nanocomposite particles, which was characterized in that a magnetic material is iron manganese, was used instead of the fluorescent material in the composite.

The components and contents of the composites of Examples 1 to 4 and Comparative Examples 1 to 4, the temperatures and retention times of the melted composites in the melting extrusion device, and whether fluorescence discrimination is possible are summarized in the following Table 1. In whether the fluorescence discrimination is possible, “o” means that the fluorescence discrimination is possible, and “x” means that the fluorescence discrimination is not possible.

TABLE 1

| | Example | | | | Comparative Example | | | |
|---|---|---|---|---|---|---|---|---|
| Items | 1 | 2 | 3 | 4 | 1 | 2 | 3 | 4 |
| (A) Polypropylene | 99 | 97 | 95 | 93 | 99 | 97 | 95 | 93 |
| (wt %) | | | | | | | | |
| (B) Fluorescent | 1 | 3 | 5 | 7 | — | — | — | — |
| material | | | | | | | | |
| (wt %) | | | | | | | | |
| (C) Magnetic | — | — | — | — | 1 | 3 | 5 | 7 |
| nanocomposite | | | | | | | | |
| particles | | | | | | | | |
| (wt %) | | | | | | | | |
| Temperature of | 200 | 210 | 220 | 230 | 200 | 210 | 220 | 230 |
| dissolution and | | | | | | | | |
| extrusion device | | | | | | | | |
| (° C.) | | | | | | | | |
| Retention time in | 5 | 10 | 15 | 20 | 5 | 10 | 15 | 20 |
| device during | | | | | | | | |
| dissolution and | | | | | | | | |
| extrusion | | | | | | | | |
| (seconds) | | | | | | | | |
| whether | ∘ | ∘ | ∘ | ∘ | x | x | x | x |
| discrimination is | | | | | | | | |
| possible | | | | | | | | |

In Table 1, (A) is polypropylene (Sigma Aldrich Co., USA) as a plastic material, (B) is 4-bis (2-benzoxazolyl) stilbene (Sigma Aldrich Co., Ltd., USA) as a fluorescent material, and (C) is magnetic nanocomposite particles containing manganese iron (Fe—Mn) as magnetic nanoparticles and a lactide-glycolide copolymer (poly (lactide-co-glycolide)) as a polymer material. In this example, the lactide-glycolide copolymer is a polymer material having a weight average molecular weight ranging from 5,000 to 20,000, and a particulate material manufactured by mixing the polymer material and manganese iron particles in a weight ratio ranging from 5 to 50 is used as (C).

In addition, in Table 1, a device of Micro compounder MC 15 HT sold by Xplore Co., Ltd., USA was used as the melting extrusion device.

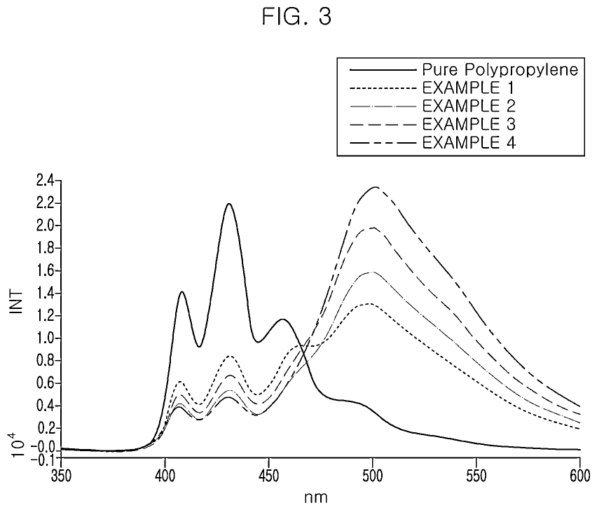

FIG. 3 is a graph showing fluorescence analysis results of the composites according to Examples 1 to 4 of the present disclosure and pure polypropylene without adding a fluorescent material.

Peak intensity ratios of the composites according to Examples 1 to 4 to a peak intensity of the pure polypropylene at a wavelength of 500 nm in the fluorescence spectrum measured as shown in FIG. 3 are shown in the following Table 2. In this example, the peak intensity ratio was calculated by dividing a peak intensity value of the composite according to each of Examples 1 to 4 at a wavelength of 500 nm by a peak intensity value of the pure polypropylene.

TABLE 2

| Items | Peak intensity ratio |
|---|---|
| Example 1 | 4.2 |
| Example 2 | 4.5 |
| Example 3 | 6.1 |
| Example 4 | 7.2 |

As shown in Table 2, it can be confirmed that, as an amount of an added fluorescent material was increased, the peak intensity ratio gradually increased, and the composite of Example 4 in which the fluorescent material was added in an amount of 7 wt % based on the total weight of the composite exhibited the highest peak intensity ratio.

The following Table 3 shows ratios of the peak intensity of the composites according to Comparative Examples 1 to 4 to the peak intensity of the pure polypropylene at a wavelength of 500 nm in the fluorescence spectrum.

TABLE 3

| Items | Peak intensity ratio |
|---|---|
| Comparative Example 1 | 1 |
| Comparative Example 2 | 1 |
| Comparative Example 3 | 1 |
| Comparative Example 4 | 1 |

Unlike Examples 1 to 4 shown in Table 2, it can be confirmed that since the fluorescent material is not added, the composites of Comparative Examples 1 to 4 exhibited the same peak intensity as that of the pure polypropylene. It can be confirmed that a large difference in fluorescence intensity was generated at a specific wavelength between the composites to which the fluorescent material was not added as in Comparative Example 1 to 4 and the composite to which the fluorescent material was added as in Examples 1 to 4 so that an eco-friendly material for a vehicle could be clearly discriminated.

As described above, the system for discriminating an eco-friendly material for a vehicle of the present disclosure scans a composite or plastic molded product to be discriminated using a fluorescence spectrometer, detects a fluorescent material included in the composite or plastic molded product, and identifies an eco-friendly material for a vehicle using simply reused waste plastic.

A detection method using a magnetic force by adding metal particles to a used plastic material as a conventional discrimination method, or a detection method of adding specific inorganic particles and burning a composite to analyze and utilize characteristics of the inorganic particles are known. However, these methods have very low discrimination reliability because a reliability deviation occurs significantly depending on the surrounding environments, and separate expensive equipment is required in a magnetic screening process for analyzing a magnetic force and inorganic particle characteristics. In addition, when a composite to which conventional metal particles or specific inorganic particles are added is to be recycled, there is a problem in that environmental pollution causes are generated in the process of removing and recovering the metal particles and inorganic particles.

However, compared to the conventional discrimination methods, the eco-friendly material discrimination method for a vehicle using the system for discriminating an eco-friendly material for a vehicle of the present disclosure has an advantage in that an eco-friendly material for discrimination is easily manufactured through melting and extrusion of a fluorescent material and plastic, the fluorescent material to be added does not affect any thermal or mechanical property change or deterioration in the composite in terms of plastic recycling, and no additional processing equipment is required.

In addition, compared to the conventional discrimination methods, the system for discriminating an eco-friendly material for a vehicle of the present disclosure has an advantage in that the analysis method is very fast and can provide highly reliable results.

While the embodiments of the present disclosure have been described, those having ordinary skill in the art can understand that the present disclosure can be implemented in other specific forms without departing from the technical spirit or the necessary features of the present disclosure. Therefore, it should be understood that the above-described embodiments are not restrictive but illustrative in all aspects.

What is claimed is:

1. A system for discriminating an eco-friendly material for a vehicle, the system comprising:
a fluorescent material supply part configured to store and supply a fluorescent material with a unique emission characteristic when irradiated with light;
a plastic supply part configured to store and supply a plastic material;
a composite compounding part configured to manufacture a composite by mixing and melting the fluorescent material supplied from the fluorescent material supply part and the plastic material supplied from the plastic supply part and extrude the manufactured composite;
a molding part configured to manufacture a plastic molded product by performing an injection molding process on the composite manufactured in the composite compounding part; and
a discrimination part configured to measure a fluorescence intensity from the composite or the plastic molded product.

2. The system of claim 1, wherein the fluorescent material includes one or more of acridine, anthracene, coumarin, cyanine, stilbene-based compounds, derivatives thereof, or combinations thereof.

3. The system of claim 1, wherein the fluorescent material is included in an amount ranging from 0.1 wt % to 10 wt % based on a total weight of the composite.

4. The system of claim 1, wherein the plastic material includes one or more of polypropylene, nylon, polycarbonate, acrylonitrile butadiene styrene, polyethylene terephthalate, or combinations thereof.

5. The system of claim 1, wherein the composite compounding part mixes and melts the fluorescent material and the plastic material at a temperature ranging from 200° C. to 270° C.

6. The system of claim 1, wherein the discrimination part measures a value of a fluorescence intensity emitted from the plastic molded product by irradiating the composite or the plastic molded product with light with a wavelength ranging from 190 nm to 900 nm.

7. The system of claim 6, wherein the discrimination part analyzes the value of the fluorescence intensity of the composite or the plastic molded product by the light irradiation.

8. A method of discriminating an eco-friendly material for a vehicle, the method comprising:
(a) irradiating a composite or a plastic molded product to be measured with light and measuring a value of a fluorescence intensity emitted therefrom; and
(b) determining whether the composite or the plastic molded product is a recycled plastic material from the measured value of the fluorescence intensity.

9. The method of claim 8, wherein, in operation (a), a value of the emitted fluorescence intensity is measured through a fluorescence spectrophotometer by irradiating the composite or the plastic molded product with light with a wavelength ranging from 190 nm to 900 nm.

10. The method of claim 8, wherein, in operation (b), whether the composite or the plastic molded product is the recycled plastic material is determined by comparing the value of the fluorescence intensity measured from the composite or the plastic molded product with a pre-stored value of a fluorescence intensity of a standard material.

11. The method of claim 10, wherein the standard material includes a plastic material not containing a fluorescent material with a unique emission characteristic when irradiated with light.

12. The method of claim 11, wherein the fluorescent material includes one or more of acridine, anthracene, coumarin, cyanine, stilbene-based compounds, derivatives thereof, or combinations thereof.

13. The method of claim 8, wherein the composite or the plastic molded product includes one or more of polypropylene, nylon, polycarbonate, acrylonitrile butadiene styrene, polyethylene terephthalate, or combinations thereof.
